(19) United States
(12) Patent Application Publication
(10) Pub. No.: US 2024/0181174 A1
(43) Pub. Date: Jun. 6, 2024

(54) PRESSURIZED AEROSOL DEVICE FOR MEDICAL PROCEDURES

(71) Applicant: ThermaSolutions, St. Paul, MN (US)

(72) Inventors: Steven James Davis, White Bear Lake, MN (US); Gregg Giza, Northfield, MN (US)

(21) Appl. No.: 18/529,996

(22) Filed: Dec. 5, 2023

Related U.S. Application Data
(60) Provisional application No. 63/386,048, filed on Dec. 5, 2022.

(51) Int. Cl. A61M 11/00

(52) U.S. Cl. CPC A61M 11/00

(57) ABSTRACT

A spray device, method, and system for delivering an aerosolized therapeutic agent to a body cavity of a patient, for example, in conjunction with administration of chemotherapeutic agents. A longitudinal spray device/nebulizer that directs an aerosolized substance in both an axial direction and a radial direction.

### CROSS-REFERENCE TO RELATED APPLICATIONS

This application claims priority to U.S. Provisional Patent Application Ser. No. 63/386,048, filed Dec. 5, 2022, the entire contents of which are incorporated by reference herein.

### TECHNICAL FIELD

In general, this disclosure is directed to devices and methods for delivering an aerosolized therapeutic agent to a body cavity of a patient.

### BACKGROUND

The treatment of certain diseases, such as cancer, that may develop in a body cavity of a patient, for example, can be complex. It would be desirable, for example, to provide relatively high doses of a chemotherapy drug directly into the affected body cavity without exposing the remainder of a patient's body to significant levels of toxicity. An example of a known surgical procedure for treating certain abdominal cancers is hyperthermic intraperitoneal chemotherapy (“HIPEC”). HIPEC may be used, for example, in conjunction with a procedure in which a surgeon accesses a patient's abdominal cavity and surgically removes cancerous tumors; HIPEC may then be used to deliver a liquid/fluid chemotherapeutic agent within the abdominal cavity of the patient. The goal of the HIPEC fluid delivery is to make contact with any yet remaining cancer cells and destroy/kill them. Heat is typically provided in conjunction with the fluid delivery during HIPEC, which may increase the efficacy of the treatment. In this way, the normal side-effects of chemotherapy can be minimized or avoided.

Minimally invasive surgery may also be performed in areas such as the abdomen by means of laparoscopy, for example. To enable the use of a laparoscopic approach, a hollow space in the body cavity of the patient is typically formed by insufflating the body cavity with a gas, typically carbon dioxide (CO2), using a CO2-insufflator, for example. Access to (and generation of) the insufflated hollow space is typically provided for via the use of trocars/trocar systems.

Recently, a technique for treating cancers and/or tumors in a body cavity of a patient, such as an abdominal cavity, has been developed. The technique employs the delivery of a pressurized aerosol chemotherapeutic agent to a body cavity, such as the peritoneum, for example. The technique may be performed using laparoscopy and/or insufflation. The technique is a relatively new method involving the application of an aerosolized chemotherapeutic agent into a body cavity of a patient.

### SUMMARY

In some embodiments, a spray device or nebulizer is disclosed for delivering an aerosolized substance into a body cavity. The spray device comprises an elongate member having a proximal portion, and a distal portion. The distal portion of the spray device is configured to be removably placed into the body cavity. The elongate member is configured to receive a substance at the proximal portion and deliver the substance from the distal portion in an aerosolized form. The distal portion of the spray device includes one or more axial spray holes configured to direct the substance from the distal portion in a generally distal direction. The distal portion of the spray device also includes one or more radial spray holes configured to direct the substance from the distal portion in an outward direction. In embodiments where the spray device is generally cylindrical, the one or more radial spray holes are configured to direct the substance from the distal portion in a radially outward direction. The axial and radial spray holes are configured to cause the substance to exit from the distal portion in an aerosolized form.

In some embodiments, a method is disclosed for delivering an aerosolized substance into a body cavity using the spray device of this disclosure. A method may include inserting a first trocar sleeve into a body cavity of a patient, inserting at least a portion of a spray device into the body cavity of the patient via the first trocar sleeve, and delivering a substance in a liquid form to a proximal portion of the spray device to thereby cause the spray device to deliver the substance to the body cavity in an aerosolized form. In some embodiments of such a method, the spray device may include a distal portion having one or more axial spray holes configured to direct the substance from the distal portion into the body cavity in a generally distal direction. The distal portion of the spray device may further include one or more radial spray holes configured to direct the substance from the distal portion into the body cavity in a radially outward direction.

In some embodiments, a system is disclosed for delivering an aerosolized substance into a body cavity using the spray device of this disclosure. A system may include a first trocar sleeve adapted to be inserted into the body cavity, a supply of insufflation gas configured to be delivered to the body cavity to form an open space or hollow space, a spray device configured to be inserted into the body cavity via the first trocar sleeve, and a supply of the substance in a liquid form coupled to a portion of the spray device, wherein the supply of the substance is capable of delivering the substance to the spray device at or above a predetermined pressure.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a simplified cross-sectional view of a body cavity of a patient in which various embodiments of this disclosure may be employed;

FIG. 2A is a perspective view of a spray device according to certain embodiments of this disclosure;

FIG. 2B is a cross-sectional view of a system for delivering an aerosolized substance to a body cavity according to certain embodiments of this disclosure;

FIGS. 3A and 3B are perspective views of exemplary embodiments of a spray device having radial spray holes spaced evenly about an outer surface of the spray device according to various embodiments;

FIGS. 4A and 4B are perspective and end views, respectively, of a distal portion of a spray device according to various embodiments;

FIGS. 5A-5I show a number of alternate arrangements of spray holes of a spray device according to various embodiments of this disclosure;

FIG. 6 is a longitudinal cross-sectional view of a distal portion of a spray device according to certain alternate embodiments of this disclosure; and

FIG. 7 is a schematic diagram of a system for delivering an aerosolized substance to a body cavity according to various embodiments of this disclosure.

### DETAILED DESCRIPTION

This disclosure relates generally to devices, methods, and systems for delivering a substance in an aerosolized form into a body cavity of a patient. This disclosure describes some embodiments of devices, methods, and systems for delivering a substance in an aerosolized form into a body cavity of a patient in both axial and radial directions during certain treatment procedures, such as during pressurized aerosol chemotherapy delivery, for example.

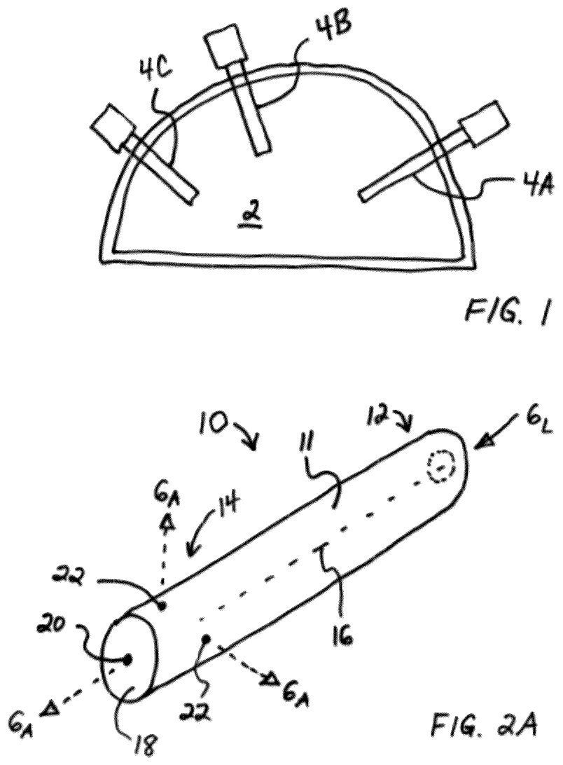

FIG. 1 is a cross-sectional view of an exemplary body cavity 2 of a patient in which various embodiments of this disclosure may be employed. Body cavity 2 may comprise a patient's abdominal cavity, as a possible example, or may comprise any other body cavity. As shown, body cavity 2 has been insufflated to form a hollow space within body cavity 2. FIG. 1 also shows a number of trocars, trocar systems, or trocar sleeves 4A, 4B, and 4C. Each trocar 4A-4C is used as a portal or access passage into the body cavity 2 to enable the placement of tools and instruments, or facilitate the supply or removal of gas or fluid to and from the body cavity 2. In some examples, a trocar such as trocar 4A may be used to enable a spray device according to this disclosure to be placed into body cavity 2. In some examples, a trocar such as trocar 4B may be used to deliver a gas, such as carbon dioxide, to the body cavity 2 to thereby insufflate the body cavity and form a hollow space.

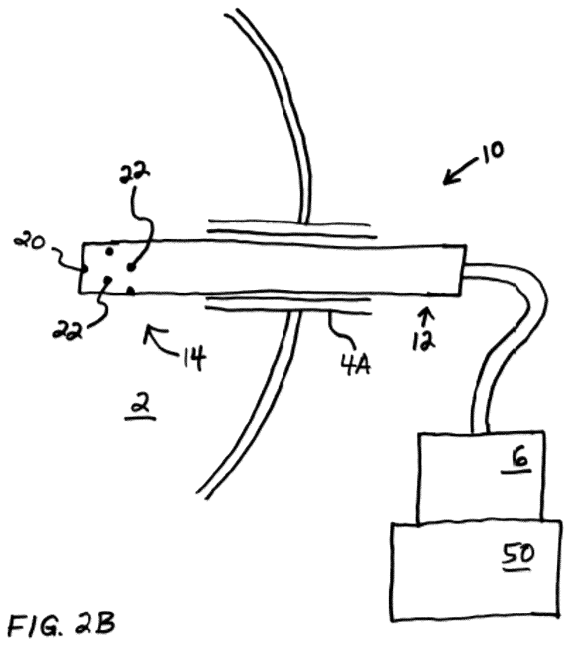

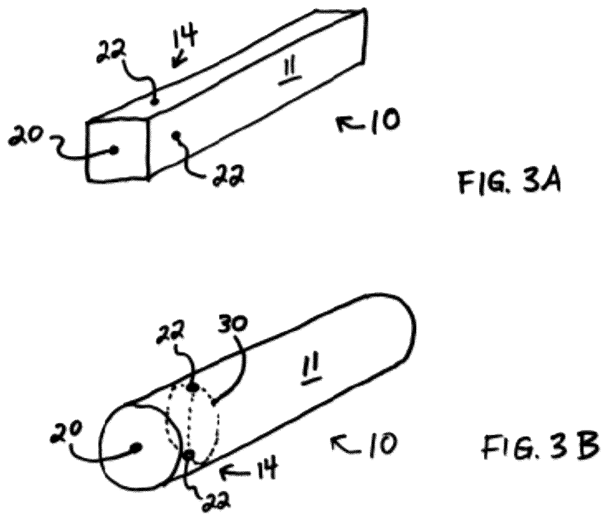

FIG. 2A is a perspective view of a spray device 10 according to certain embodiments of this disclosure. Spray device 10 comprises an elongate member 11 having a proximal portion 12, a distal portion 14, and a longitudinal axis 16 extending generally from the proximal portion 12 to the distal portion 14. It is noted that elongate member 11 need not be perfectly straight as shown in the drawing figures; elongate member 11 may include some curvature or a bend, for example, to possibly facilitate positioning of the distal portion 14 within the body cavity 2. In such an embodiment, longitudinal axis 16 could, for example, follow the contour of the elongate member 11 from the proximal portion 12 to the distal portion 14 and need not be straight. The distal portion 14 of the spray device 10 is configured to be removably placed in the body cavity 2; this may be done, for example, via a trocar such as trocar sleeve 4A. Spray device 10 is configured to receive a substance 6 (typically in a liquid form 61) in an opening at the proximal portion 12. The substance 6 may be supplied or delivered to the spray device 10 at a pressure. The elongate member 11 of the spray device 10 is configured to deliver the substance 6 in an aerosolized form 6A from the distal portion 14 of the spray device 10. For example, during certain procedures, the substance 6 is delivered in an aerosolized form 6A into the body cavity 2.

Substance 6 may, for example, comprise a drug suitable for chemotherapy. Substance 6 may be provided and supplied to the spray device 10 in a liquid form, and then delivered into a body cavity, such as the peritoneal cavity, for example, in an aerosol form. Drugs referred to as cytostatics, such as doxorubicin, cisplatin, and/or other chemotherapeutic agents are examples of such drugs which may be used as substance 6 in conjunction with spray device 10.

In certain embodiments, the distal portion 14 of the spray device 10 may include one or more axial spray holes 20 configured to direct the substance 6 from the distal portion 14 in a distal direction, e.g., generally parallel to the longitudinal axis 16 of the elongate member 11. The size of the axial spray hole(s) 20, in combination with a pressurized supply of the substance 6 at the proximal portion 12, may cause the substance 6 to exit from the axial spray hole(s) 20 in an aerosolized form 6A. Additionally, the distal portion 14 of the spray device 10 may include one or more radial spray holes 22 configured to direct the substance 6 from the distal portion 14 in a radially outward direction, e.g., generally perpendicular to the longitudinal axis 16 of the elongate member 11. The size of the radial spray hole(s) 22, in combination with a pressurized supply of the substance 6 at the proximal portion 12, may cause the substance 6 to exit from the radial spray hole(s) 22 in an aerosolized form 6A.

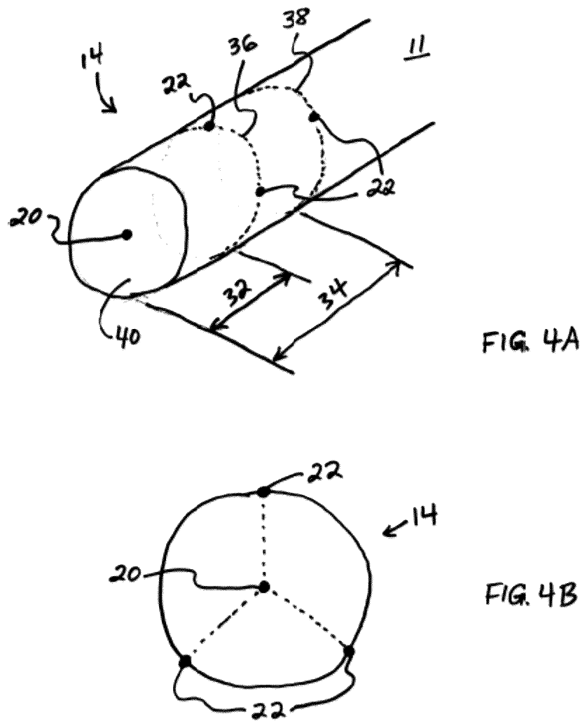

Spray device 10 may have at least two radial spray holes 22 disposed in the distal portion 14 of the elongate member 11 according to some embodiments. For example, FIG. 2 shows spray device 10 having two radial spray holes 22. FIGS. 3A and 3B show exemplary embodiments of a distal portion 14 of a spray device 10 having two radial spray holes 22 spaced evenly about an outer surface of the elongate member 11. FIG. 3A shows an embodiment where the outer surface of elongate member 11 is square or rectangular shaped, whereas FIG. 3B shows an embodiment where the outer surface of elongate member 11 is generally round (e.g., circular, oval, etc.). In embodiments where the elongate member 11 of spray device 10 has a generally round outer surface, as in FIG. 3B, the radial spray holes 22 may be evenly spaced about a circumference 30 of the distal portion 14 of the elongate member 11 as illustrated in FIG. 3B.

Spray device 10 may be configured such that the axial spray hole(s) 20 and/or the radial spray holes 22 cause the substance 6 to exit from the distal portion 14 of the elongate member 11 in the aerosolized form when the substance 6 is received at the proximal portion 12 in the liquid form at or above a certain pressure. The size of the various spray holes, in combination with a sufficiently pressurized supply of the substance 6 at the proximal portion 12, may cause the substance 6 to exit from the spray holes in an aerosolized form. The cross-sectional shape of the spray holes (e.g., nozzle, venturi, etc.) may also facilitate aerosolization of the substance 6 as it moves through the spray holes. The use of axial spray hole(s) 20 and radial spray holes 22 to cause the substance 6 to exit from the distal portion 14 in an aerosolized form may facilitate and/or enhance the distribution and/or application of the substance 6 in the body cavity 2, for example, to more effectively deliver a therapeutic agent in the body cavity 2.

In some embodiments, substance 6 may be delivered from spray device 10 through axial spray hole(s) 20 and radial spray holes 22 to achieve a certain desired flow rate of substance 6 into the body cavity 2 of the patient. For example, substance 6 may be supplied (e.g., under pressure from a supply source) so that the volumetric flow rate of substance 6 into body cavity 2 is greater than about 10 mL/min, according to some embodiments. In further embodiments, substance 6 may be supplied at a volumetric flow rate that falls within a range of from 20 mL/min to 120 mL/min, and in certain embodiments, substance 6 may be delivered into body cavity 2 of a patient at a volumetric flow rate between about 30 mL/min and about 60 mL/min. These values and ranges are, of course, exemplary only and those of ordinary skill in the art would be able to modify the flow rates to suit a particular application with the benefit of these teachings.

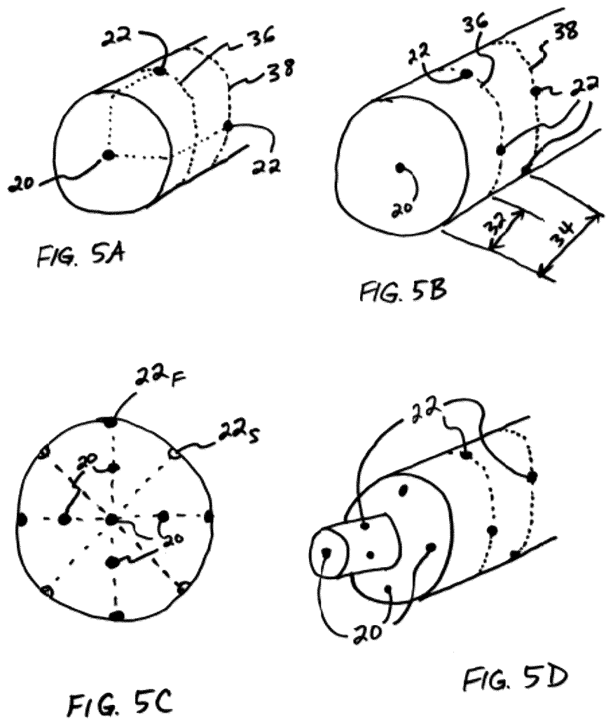

In FIG. 4A, elongate member 11 of spray device 10 has a distal portion 14 comprising four radial spray holes spaced 90 degrees about the circumference of the distal portion 14 of the elongate member 11. In an alternative embodiment, FIG. 4B shows an arrangement of three radial spray holes spaced 120 degrees about the circumference of the distal portion 14 of the elongate member 11. Other equivalent permutations and arrangements would be apparent to one of ordinary skill in the art.

In FIGS. 4A and 4B, a single axial spray hole 20 is shown disposed at a distal end 40 of the elongate member 11. However, one or more axial spray holes 20 may be disposed in the distal portion 14 of the elongate member 11, and they may be, for example, disposed somewhat proximal to the distal end 40 (e.g., they may formed in a recessed tip proximal of the distal end 40. With further reference to FIG. 4A, a first row 36 of at least two radial spray holes 22 may be located or disposed at a first axial distance 32 from a distal end 40 of the elongate member 11. In some embodiments, a second row 38 comprising at least two radial spray holes 22 may be disposed at a second axial distance 34 from the distal end 40 of the elongate member 11, as seen in FIG. 4A. The radial spray holes 22 of the second row 38 may also be evenly spaced about the circumference 30 of the distal portion 14 of the elongate member 11. Additional rows of radial spray holes are contemplated by this disclosure, which may facilitate distribution of the substance 6 in the body cavity 2 of a patient.

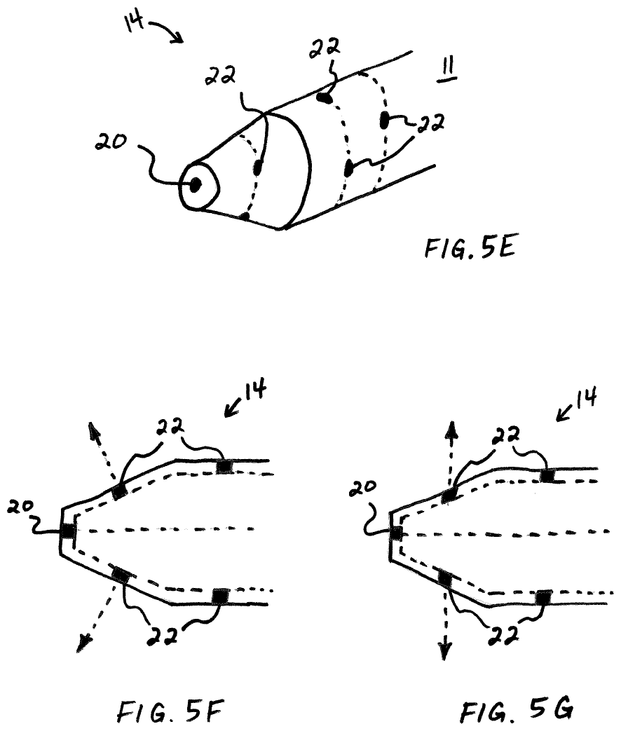

In some embodiments, spray device 10 may have a distal portion 14 in which there is a first row 36 and a second row 38 of radial spray holes 22, and wherein the number of radial spray holes 22 in the first row 36 is equal to the number of radial spray holes 22 in the second row 38. For example, FIG. 5A shows an embodiment in which the first and second rows have two radial spray holes each. As shown in FIG. 5A, the spray holes 22 of the second row 38 may be angularly offset from the spray holes 22 of the first row 36 in some embodiments; this may, for example, facilitate a more even distribution of the substance 6 in the body cavity 2.

FIG. 5B shows an exemplary spray device 10 according to some embodiments in which the first row 36 comprises four radial spray holes 22, and the second row 38 comprises four radial spray holes 22. In the example shown in FIG. 5B, the spray holes 22 in the second row 38 are angularly offset from the spray holes 22 in the first row 36. In some preferred embodiments, the four radial spray holes 22 of the second row 38 are angularly offset from the four radial spray holes 22 of the first row 36 by 45 degrees. This is perhaps better seen in FIG. 5C showing a distal end view of a spray device 10 showing the angular disposition of the spray holes in the first and second rows 36, 38. For example, the four radial spray holes 22F of the first row 36 are disposed at the 3, 6, 9 and 12 o'clock positions, while the four radial spray holes 22s of the second row 38 are each disposed 45 degrees offset from the positions of the spray holes of the first row 36. It should also be noted that FIG. 5C shows an embodiment having more than one axial spray hole 20. For example, FIG. 5C shows five axial spray holes 20 in a distal end of elongate member 11.

FIG. 5D shows an alternate embodiment that would be contemplated by one of ordinary skill in the art and would be deemed within the scope of the claims presented. For example, FIG. 5D shows a spray device having more than one axial spray hole 20, some of which are disposed proximally of the distal end 40 of the elongate member 11. In this particular embodiment, there are three rows of radial spray holes 22 as shown. FIGS. 5E-5G show another alternate embodiment that would be contemplated by one of ordinary skill in the art and would be deemed within the scope of the claims presented. For example, FIG. 5E is a partial perspective view showing a distal portion 14 of a spray device having a tapered shape (e.g., a conical shape or a parabolic shape). In the embodiment depicted in FIGS. 5E-5G, there are two or more radial spray holes 22 disposed along the tapered portion of the distal portion 14 of the spray device. FIGS. 5F and 5G show that the radial spray holes 22 disposed along the tapered portion may be directed in different ways. For example, in the cross-sectional side view of FIG. 5F, the radial spray holes 22 may be formed to direct substance 6 outwardly from the tapered surface in a direction generally perpendicular to the tapered surface, as shown in FIG. 5F. Alternatively, in the cross-sectional side view of FIG. 5G, the radial spray holes 22 may be formed to direct substance 6 outwardly from the tapered surface in a direction generally perpendicular to the axis of the elongate spray device, as shown in FIG. 5G. FIGS. 5H and 5I show yet further alternative embodiments in which there may be radial spray holes 22 disposed along the tapered portion, and an axial spray hole 20 at the distal end, but no radial spray holes disposed along the cylindrical portion of the elongate member 11. FIG. 5H shows a single row of such radial spray holes 22, while FIG. 5I shows two rows of radial spray holes 22, as possible examples of such embodiments. Further modifications would be apparent to one of ordinary skill in the art with the benefit of these teachings.

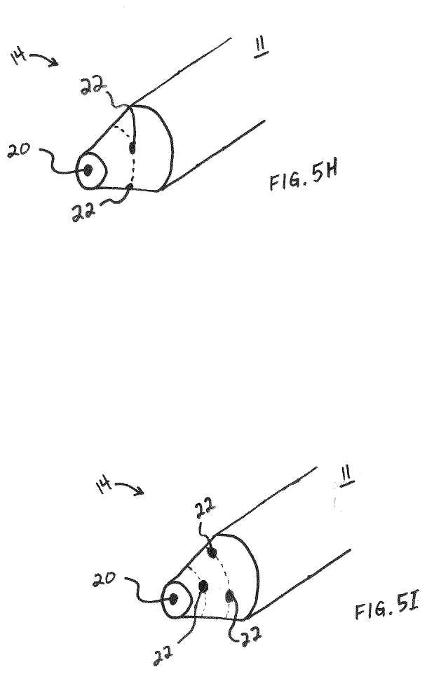

FIG. 6 is a longitudinal cross-sectional view of a distal portion 14 of a spray device 10 according to certain embodiments. FIG. 6 shows at least one (and in this example, two) radial spray holes 22 angled towards a proximal portion of the elongate member 11. That is, the openings for these radial spray holes are formed so that substance 6, when delivered from distal portion 14 under pressure, will be directed both outwardly from the elongate member 11 and proximally of the distal end 40. This may have the effect of counteracting or offsetting any axially-directed force (e.g., a recoil force) caused by the substance 6 being directed from the one or more axial spray holes 20, for example. The angled spray holes 22 of FIG. 6 may also (or alternately) facilitate and/or enhance the evenness of the distribution of substance 6 within the body cavity 2 of the patient. For example, in some embodiments, there may be two rows of angled spray holes 22 wherein the row of spray holes 22 that is more distally disposed might be angled toward the distal end of spray device 10 (not shown), and the row of spray holes 22 that is more proximally disposed might be angled proximally (as shown in FIG. 6) to further facilitate the even distribution of substance 6 in body cavity 2.

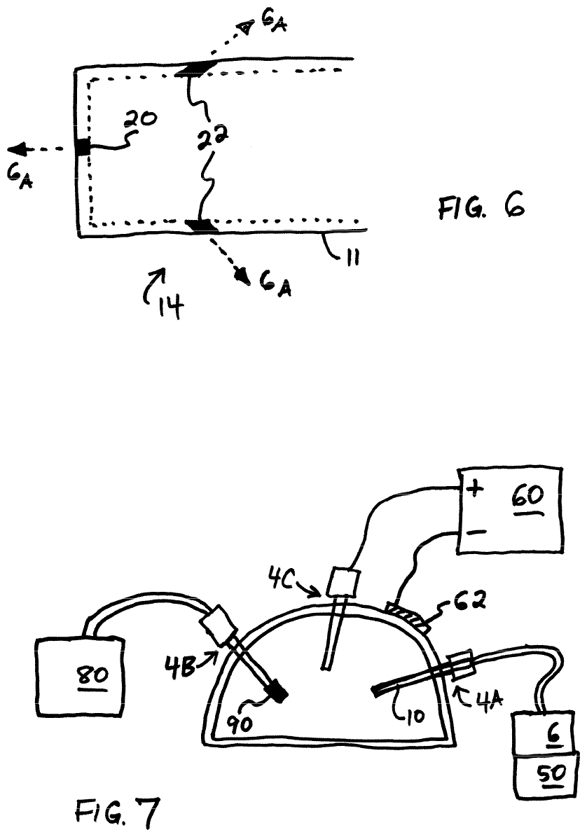

The function of spray device 10 may be further enhanced during certain procedures by creating an electric field within the body cavity 2. For example, FIG. 7 shows an example of spray device 10 being used in body cavity 2 where an electrical energy source 60 is provided to deliver an electrostatic charge to the body cavity 2 of the patient to thereby alter the spray path of an aerosolized agent (e.g., substance 6A) delivered to the body cavity by the spray device 10. For example, electrical energy source 60 may be configured to produce an electrical field in the body cavity. This may be done, for example, by applying a voltage between the distal portion 14 of the elongate member 11 and an area of skin of the patient (e.g., via an electrode 62 applied to a surface of the patient's skin, as shown in FIG. 7). An electrical field may be thereby generated to facilitate distribution of the substance 6 in the aerosolized form in the body cavity 2. In some embodiments, electrical energy source 60 may generate an electrical field in the body cavity 2 via a third trocar sleeve 4C inserted into the body cavity 2. In some embodiments, the electrical energy source 60 may be configured to apply a voltage between the first trocar sleeve 4A and the third trocar sleeve 4C to generate an electric field.

A system for performing aerosolized chemotherapy delivery procedures is disclosed herein. Such a system may be useful for delivering a substance 6 (e.g., a chemotherapeutic agent) in an aerosolized form to a body cavity 2 of a patient. The system may include a first trocar sleeve adapted to be inserted into the body cavity 2 of a patient to enable access to the body cavity 2 by tools, instruments, etc. The system may further include a supply 80 of insufflation gas (e.g., carbon dioxide gas) configured to be delivered into the body cavity 2 to form a hollow space within body cavity 2.

The system may further include a spray device 10 as described herein, the spray device 10 being configured to be inserted into the hollow space formed within the body cavity 2 via the first trocar sleeve 4A. The spray device 10 may comprise an elongate member 11 having a proximal portion 12, a distal portion 14, and a longitudinal axis 16 extending generally from the proximal portion 12 to the distal portion 14. The elongate member 11 may be configured to receive the substance 6 in a liquid form at the proximal portion 12. The elongate member 11 may further be configured to deliver the substance 6 to the body cavity 2 in an aerosolized form from a number of spray holes formed in the distal portion 14 of the elongate member 11. The spray holes may include one or more axial spray holes 20 configured to direct the substance 6 from the distal portion 14 in a generally distal direction. The spray holes may further include one or more radial spray holes 22 configured to direct the substance 6 from the distal portion 14 in a generally radially outward direction. The one or more axial spray holes 20 and the one or more radial spray holes 22 are configured to cause the substance 6 to exit from (or flow from, or spray from) the distal portion 14 in an aerosolized form.

The system may further include a source or supply 50 of the substance 6 in a liquid form. The supply/source 50 of the substance 6 is configured to be operably coupled to the proximal portion 12 of the elongate member 11. Source 50 may be configured to deliver the substance 6 to the proximal portion 12 of the elongate member 11 at a predetermined fluid flow rate, for example. In some embodiments, source 50 may be configured to deliver the substance 6 to the proximal portion 12 of the elongate member 11 to thereby facilitate delivery of substance 6 to a body cavity 2 of the patient at a fluid flow rate that is greater than 10 mL/min, or that falls within a defined range, such as between 20 mL/min and 120 mL/min, or in certain cases, between about 30 mL/min and about 60 mL/min, according to various embodiments. Alternately, the source 50 may be configured to deliver the substance 6 to the proximal portion 12 of the elongate member 11 at or above a predetermined pressure, for example. Source 50 may, for example, comprise a pump for supplying substance 6 at a pressure to proximal portion 12. In other embodiments, source 50 may comprise a syringe/piston arrangement to pressurize substance 6 for delivery, or may be configured to pressurize substance 6 using pneumatic or hydraulic means, or using other means known to those of ordinary skill in the art.

FIG. 2B shows a system according to some embodiments. A system for performing aerosolized chemotherapy delivery procedures may include a second trocar sleeve 4B different from the first trocar sleeve 4A. The second trocar sleeve 4B is adapted to be inserted into the body cavity 2 at a different location from the first trocar sleeve 4A. An insufflation gas may be delivered from an insufflation gas supply 80 into the body cavity 2 via the second trocar sleeve 4B to form a hollow space. In certain embodiments, a leak detector may be included as part of the system to detect leakage of insufflation gas from the body cavity. For example, a sensor configured to sense or detect a leak of insufflation gas (e.g., a CO2 sensor) may be disposed on or near the first trocar sleeve 4A (e.g., the trocar used to position at least a portion of the spray device into the body cavity).

A system for performing aerosolized chemotherapy delivery procedures may, in some embodiments, further include an electrical energy source 60 configured to generate an electrical field in the body cavity 2. For example, the electrical energy source 60 may apply a voltage between a portion of the elongate member 11 (e.g., the distal portion 14) and an area of skin of the patient (e.g., an external skin surface). The electrical field that is thereby generated may facilitate distribution of the substance 6 in an aerosolized form in the body cavity 2. In some examples, the electrical energy source 60 may generate the electrical field in the body cavity 2 via a third trocar sleeve 4C adapted to be inserted into the body cavity 2. The electrical energy source 60 may be configured to apply a voltage between the first trocar sleeve 4A and the third trocar sleeve 4C to generate the electric field according to some embodiments.

A method of performing aerosolized chemotherapy delivery procedures is disclosed herein. Such a method may be useful for delivering a substance 6 (e.g., a chemotherapeutic agent) in an aerosolized form to a body cavity 2 of a patient. For example, the method may include inserting a first trocar sleeve into a body cavity 2 of a patient, inserting at least a portion of a spray device 10 into the body cavity 2 via the first trocar sleeve 4A, and delivering a substance 6 in an aerosolized form from a distal portion 14 of the spray device 10. Such a method may include using a spray device 10 that includes an elongate member 11 having a proximal portion 12, a distal portion 14, and a longitudinal axis 16 extending generally from the proximal portion 12 to the distal portion 14. The elongate member 11 is configured to receive the substance 6 in a liquid form at the proximal portion 12. The elongate member 11 of the spray device 10 is further configured to deliver the substance 6 to the body cavity 2 in an aerosolized form from a number of spray holes formed in the distal portion 14 of the elongate member 11. For example, the distal portion 14 of the elongate member 11 may have at least one axial spray hole 20 configured to direct the substance 6 from the distal portion 14 in a generally distal direction. The distal portion 14 of the elongate member 11 may further have at least one radial spray hole 22 configured to direct the substance from the distal portion 14 in a generally radially outward direction. The axial spray hole(s) and the radial spray hole(s) are designed/configured to cause the substance 6 to move through the spray holes and exit from the distal portion 14 in an aerosolized form of the substance 6.

The method may, for example, further comprise supplying the substance 6 to the proximal portion 12 of the elongate member 11. In some embodiments, supplying the substance 6 includes delivering substance 6 from a source 50 at a pressure that is high enough to cause the substance to exit through the spray holes of the distal portion 14 in an aerosolized form. The substance 6 may be delivered to the proximal portion 12 in a liquid form according to some embodiments. The substance 6 may be delivered in the liquid form to the proximal portion 12 at or above a predetermined pressure to thereby cause the substance 6 to be delivered in the aerosolized form to the body cavity 2. In some embodiments, substance 6 may be delivered in the liquid form to the proximal portion 12 at a predetermined flow rate, or within a predefined flow rate range (e.g., between 20 mL/min and 120 mL/min, or in some cases, between about 30 mL/min and about 60 mL/min) to thereby cause the substance 6 to be delivered in the aerosolized form to the body cavity 2.

The method may, for example, further comprise supplying an insufflation gas to the body cavity 2. For example, a supply 80 of insufflation gas (e.g., carbon dioxide gas) may be configured to deliver the insufflation gas into the body cavity 2 to form a hollow space within body cavity 2. In some embodiments, this step may be preceded by inserting a second trocar sleeve 4B into the body cavity 2, and then using the second trocar sleeve 4B as the access point through which the insufflation gas is delivered to the body cavity 2. In some embodiments, second trocar sleeve 4B and first trocar sleeve 4A are different trocars disposed at two distinct locations; alternately, a single trocar could be used for both insufflation of the body cavity 2 and for positioning of the spray device 10 within the body cavity 2. In some embodiments, the aerosolized form of the substance 6 is delivered to the hollow space formed in the body cavity 2 by the supply of insufflation gas.

In some embodiments, the method may include using a spray device 10 comprising a number of spray holes in a distal portion 14 of the spray device 10. For example, distal portion 14 of elongate member 11 may include at least two radial spray holes 22 disposed at a first axial distance 32 from a distal end 40 of the elongate member 11. The two or more radial spray holes 22 so positioned may form a first row 36 of radial spray holes 22 evenly spaced about an outer surface (e.g., about a circumference in the case of a circular outer surface) of the distal portion 14 of the elongate member 11.

In some further embodiments, the method may include using a spray device 10 comprising at least two radial spray holes 22 disposed at a second axial distance 34 from the distal end 40 of the elongate member 11. The two or more radial spray holes 22 so positioned may form a second row 38 of radial spray holes 22 evenly spaced about an outer surface (e.g., about a circumference in the case of a circular outer surface) of the distal portion 14 of the elongate member 11. In some additional embodiments, the radial spray holes 22 of the second row 38 may be angularly offset from the radial spray holes 22 of the first row 36.

In certain embodiments, the method may further comprise inserting a camera 90 into the body cavity 2 via the second trocar sleeve 4B. In some embodiments, the step of inserting the camera 90 into the body cavity 2 via the second trocar sleeve 4B occurs prior to the step of delivering an insufflation gas to the body cavity 2 via the second trocar sleeve 4B.

In some embodiments, the method may further comprise installing and/or placing a sensor to detect a leak of the insufflation gas from the body cavity 2. For example, if carbon dioxide is used as the insufflation gas, a carbon dioxide sensor may be placed on, at, or near a trocar sleeve (e.g., the first trocar sleeve 4A, or optionally, the third trocar sleeve 4C in embodiments in which a third trocar sleeve 4C is used for example to establish an electric field within the body cavity 2). In such an embodiment where a leak sensor is employed, an additional step to the method may comprise monitoring the first trocar sleeve 4A for leakage of insufflation gas from the body cavity 2.

In some embodiments, a method may include the following steps. Two trocars (e.g., first trocar 4A and second trocar 4B) are inserted into a patient to provide access to the body cavity 2 of the patient. First trocar 4A is used to enable at least the distal portion 14 of spray device 10 to enter the body cavity 2 of the patient, and second trocar 4B is used to enable the supply of an insufflation gas (e.g., carbon dioxide) to the body cavity 2. In some embodiments, a camera 90 may be inserted into the patient through the second trocar 4B (e.g., the same trocar used for the insufflation gas supply). The spray device 10 is secured to the first trocar 4A, with the distal portion 14 extending into the patient. Insufflation gas (e.g., carbon dioxide) is supplied into the body cavity 2 of the patient through second trocar 4B to form a space within the body cavity 2 of the patient.

During the insufflation process, a sensor is monitored to ensure that there is no leakage of the insufflation gas, e.g., carbon dioxide, from the patient. The leak sensor or detector may be configured to detect the particular gas (e.g., a carbon dioxide sensor, if using carbon dioxide as the insufflation gas). Such a sensor may be disposed, for example, at the first trocar 4A, or along the spray device 10, or within the spray device 10, for example.

The spray device 10 is activated to deliver a substance 6 in an aerosolized form from the distal portion 14 of the spray device 10 into the space formed in the body cavity 2. The substance 6 may be a therapeutic agent, such as a medication or a chemotherapy drug in some exemplary embodiments. The spray device 10 may, for example, receive a chemotherapy drug in a liquid form under pressure at a proximal portion 12 of the spray device 10, and deliver it from the distal portion 14 of the spray device 10 in an aerosolized form to the body cavity 2 of the patient.

In some embodiments, the used or waste aerosolized substance 6 may be evacuated from the patient's body cavity 2 to a filtered waste system through a trocar. In some embodiments, the used or waste aerosolized substance 6 may be evacuated from the patient's body cavity 2 via the first trocar 4A, or alternately, via a third trocar 4C according to some embodiments.

In some embodiments, a method of performing an aerosolized chemotherapy delivery procedure may further include an optional/additional step of delivering an electrostatic charge to the body cavity 2 of the patient to alter or modify the spray path of an aerosolized substance 6 delivered to the body cavity 2 by the spray device 10.

Various examples have been described. These and other examples are within the scope of the following claims.

What is claimed is:

1. A spray device for delivering an aerosolized substance into a body cavity, the spray device comprising:
an elongate member having a proximal portion, a distal portion, and a longitudinal axis extending generally from the proximal portion to the distal portion, the distal portion being configured to be removably placed in the body cavity, the elongate member configured to receive a substance in a liquid form at the proximal portion, the elongate member further configured to deliver the substance to the body cavity in an aerosolized form from the distal portion, the distal portion comprising:
one or more axial spray holes configured to direct the substance from the distal portion in a distal direction; and
one or more radial spray holes configured to direct the substance from the distal portion in a radially outward direction,
the one or more axial spray holes and the one or more radial spray holes configured to cause the substance to exit from the distal portion in the aerosolized form.

2. The spray device of claim 1 wherein the elongate member includes at least two radial spray holes disposed in the distal portion of the elongate member, the at least two radial spray holes being spaced evenly about an outer surface of the elongate member.

3. The spray device of claim 2 wherein the elongate member has a generally round outer surface, and wherein the at least two radial spray holes are evenly spaced about a circumference of the distal portion of the elongate member.

4. The spray device of claim 2 wherein the one or more axial spray holes and the one or more radial spray holes are configured to cause the substance to exit from the distal portion in the aerosolized form when the substance is received at the proximal portion in the liquid form at a flow rate of between 20 ml/min and 120 mL/min.

5. The spray device of claim 3 wherein the elongate member comprises four radial spray holes spaced 90 degrees about the circumference of the distal portion of the elongate member.

6. The spray device of claim 3 wherein the elongate member comprises three radial spray holes spaced 120 degrees about the circumference of the distal portion of the elongate member.

7. The spray device of claim 3 wherein the at least two radial spray holes are disposed at a first axial distance from a distal end of the elongate member to form a first row of radial spray holes.

8. The spray device of claim 7 wherein one axial spray hole is disposed at the distal end of the elongate member.

9. The spray device of claim 7 wherein one axial spray hole is disposed proximal to the distal end of the elongate member.

10. The spray device of claim 7 further comprising a second row of radial spray holes disposed at a second axial distance from the distal end of the elongate member, the second row comprising at least two radial spray holes evenly spaced about the circumference of the distal portion of the elongate member.

11. The spray device of claim 10 wherein a number of radial spray holes in the first row is equal to a number of radial spray holes in the second row.

12. The spray device of claim 11 wherein the first row comprises 4 radial spray holes and the second row comprises 4 radial spray holes, and wherein the 4 radial spray holes of the second row are angularly offset from the 4 radial spray holes of the first row by 45 degrees.

13. The spray device of claim 1 wherein at least one of the at least two radial spray holes is angled towards the proximal portion of the elongate member.

14. A method for delivering a substance in an aerosolized form to a body cavity of a patient, the method comprising:
inserting a first trocar sleeve into the body cavity;
inserting at least a portion of a spray device into the body cavity via the first trocar sleeve, the spray device comprising:
an elongate member having a proximal portion, a distal portion, and a longitudinal axis extending generally from the proximal portion to the distal portion, the elongate member configured to receive the substance in a liquid form at the proximal portion, the elongate member further configured to deliver the substance to the body cavity in the aerosolized form from the distal portion, the distal portion of the elongate member comprising:
one or more axial spray holes configured to direct the substance from the distal portion in a distal direction; and
one or more radial spray holes configured to direct the substance from the distal portion in a radially outward direction,
the one or more axial spray holes and the one or more radial spray holes configured to cause the substance to exit from the distal portion in the aerosolized form; and
delivering the substance in the liquid form to the proximal portion of the elongate member to thereby cause the substance to be delivered in the aerosolized form from the distal portion of the elongate member to the body cavity.

15. The method of claim 14 further comprising:
prior to delivering the substance in the liquid form to the proximal portion of the elongate member to thereby cause the substance to be delivered in the aerosolized form to the body cavity:
inserting a second trocar sleeve into the body cavity; and
delivering an insufflation gas via the second trocar sleeve to form a hollow space in the body cavity,
wherein the substance is delivered in the aerosolized form to the hollow space formed in the body cavity.

16. The method of claim 15 wherein the substance is delivered in the liquid form to the proximal portion of the elongate member at a flow rate between 20 mL/min and 120 mL/min to thereby cause the substance to be delivered in the aerosolized form to the body cavity.

17. The method of claim 15 wherein the distal portion of the elongate member further comprises:
at least two radial spray holes disposed at a first axial distance from a distal end of the elongate member to form a first row of radial spray holes evenly spaced about a circumference of the distal portion of the elongate member; and
at least two radial spray holes disposed at a second axial distance from the distal end of the elongate member to form a second row of radial spray holes evenly spaced about the circumference of the distal portion of the elongate member,
wherein the radial spray holes of the second row are angularly offset from the radial spray holes of the first row.

18. The method of claim 15 further comprising:
prior to delivering the insufflation gas via the second trocar sleeve,
inserting a camera into the body cavity via the second trocar sleeve.

19. The method of claim 15 further comprising:
monitoring the first trocar sleeve for leakage of insufflation gas from the body cavity.

20. A system for delivering a substance in an aerosolized form to a body cavity of a patient, the system comprising:
a first trocar sleeve adapted to be inserted into the body cavity;
a supply of insufflation gas configured to be delivered to the body cavity to form a hollow space within the body cavity; and
a spray device configured to be inserted into the hollow space within the body cavity via the first trocar sleeve, the spray device comprising:
an elongate member having a proximal portion, a distal portion, and a longitudinal axis extending generally from the proximal portion to the distal portion, the elongate member configured to receive the substance in a liquid form at the proximal portion, the elongate member further configured to deliver the substance to the body cavity in the aerosolized form from the distal portion, the distal portion of the elongate member comprising:
one or more axial spray holes configured to direct the substance from the distal portion in a distal direction; and
one or more radial spray holes configured to direct the substance from the distal portion in a radially outward direction,
the one or more axial spray holes and the one or more radial spray holes configured to cause the substance to exit from the distal portion in the aerosolized form,
and
a supply of the substance in the liquid form operably coupled to the proximal portion of the elongate member and configured to deliver the substance to the proximal portion at a flow rate between 20 ml/min and 120 ml/min.

21. The system of claim 20 further comprising:
a second trocar sleeve different from the first trocar sleeve, the second trocar sleeve adapted to be inserted into the body cavity,
wherein the supply of insufflation gas is configured to be delivered to the body cavity via the second trocar sleeve to form the hollow space.

22. The system of claim 21 further comprising:
an electrical energy source configured to generate an electrical field in the body cavity.

23. The system of claim 22 wherein the electrical energy source is configured to apply a voltage between the distal portion of the elongate member and an area of skin of the patient, and wherein the electrical field is thereby generated to facilitate distribution of the substance in the aerosolized form in the body cavity.

24. The system of claim 22 wherein the electrical energy source generates the electrical field in the body cavity via a third trocar sleeve adapted to be inserted into the body cavity.

25. The system of claim 24 wherein the electrical energy source is configured to apply a voltage between the first trocar sleeve and the third trocar sleeve.
